(19) United States
(12) Patent Application Publication
(10) Pub. No.: US 2008/0097317 A1
(43) Pub. Date: Apr. 24, 2008

(54) Infusion pump

(76) Inventors: Jeffery Alholm, Indianapolis, IN (US); Fredric B. Grossman, Fishers, IN (US); Chad L. Beach, Littleton, CO (US)

Correspondence Address: BAKER & DANIELS LLP, 300 NORTH MERIDIAN STREET, SUITE 2700, INDIANAPOLIS, IN, 46204, US

(21) Appl. No.: 11/510,320

(22) Filed: Aug. 25, 2006

(51) Int. Cl. A61M 1/00

(52) U.S. Cl. 604/131

(57) ABSTRACT

A device for providing a plurality of agent delivery systems via a single housing.

### FIELD OF THE INVENTION

The present invention relates generally to the administration of liquid agents to patients. More particularly, the present invention relates to a device and method that provides portable and disposable infusion pumps for various agents and conditions.

### BACKGROUND AND SUMMARY

Drug delivery devices are used to infuse medications or other biologically active substances into human or animal subjects. For administration of selected substances, a substantially constant level of the substance is desired over an extended period. Accordingly, while a large initial bolus dose may be used in conjunction with the extended dose, the initial bolus dose is typically unsuitable for achieving an extended dose by itself. Furthermore, applications arise that require the administration of more than one substance to a particular patient or administration of one substance at multiple locations.

Accordingly, a device and method are provided that allow the constant administration of multiple substances over defined periods of time and administration of the same substance at multiple locations over defined periods of time.

According to one aspect of the present invention, an infusion pump is provided. The pump comprising a housing; a first reservoir disposed within the housing; and a second reservoir disposed within the housing. The first reservoir is part of a first agent delivery system, the second reservoir is part of a second agent delivery system, and the first agent delivery system is discreet and fluidly isolated from the second agent delivery system.

According to another aspect of the present invention, an infusion pump is provided. The pump comprising a housing; means coupled to the housing for selectively holding and dispensing a first agent; means coupled to the housing for selectively holding and dispensing a second agent; and means for selectively coupling the housing to an article of clothing.

According to another aspect of the present invention, a method of providing a plurality of agents to a patient is provided. The method including the steps of providing a housing; providing a first reservoir within the housing; providing a second reservoir within the housing; determining a desired egress flow rate for the first reservoir; determining a desired egress flow rate for the second reservoir; providing a first tubing set coupled to the first reservoir configured to provide the egress flow characteristics determined in the first reservoir egress determining step; and providing a second tubing set coupled to the second reservoir configured to provide the egress flow characteristics determined in the second reservoir egress determining step.

Additional features of the present invention will become apparent to those skilled in the art upon consideration of the following detailed description of the presently perceived best mode of carrying out the invention.

### BRIEF DESCRIPTION OF THE DRAWINGS

The detailed description of the drawings particularly refers to the accompanying figures in which:

FIG. 1 is a perspective view of an infusion pump; and

FIG. 2 is an exploded view of the infusion pump of FIG. 1.

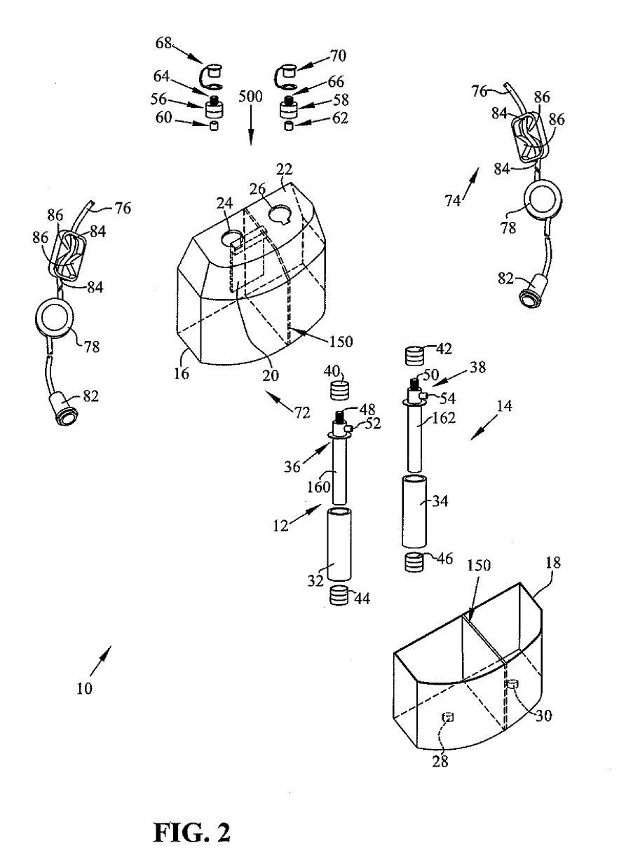

### DETAILED DESCRIPTION OF THE DRAWINGS

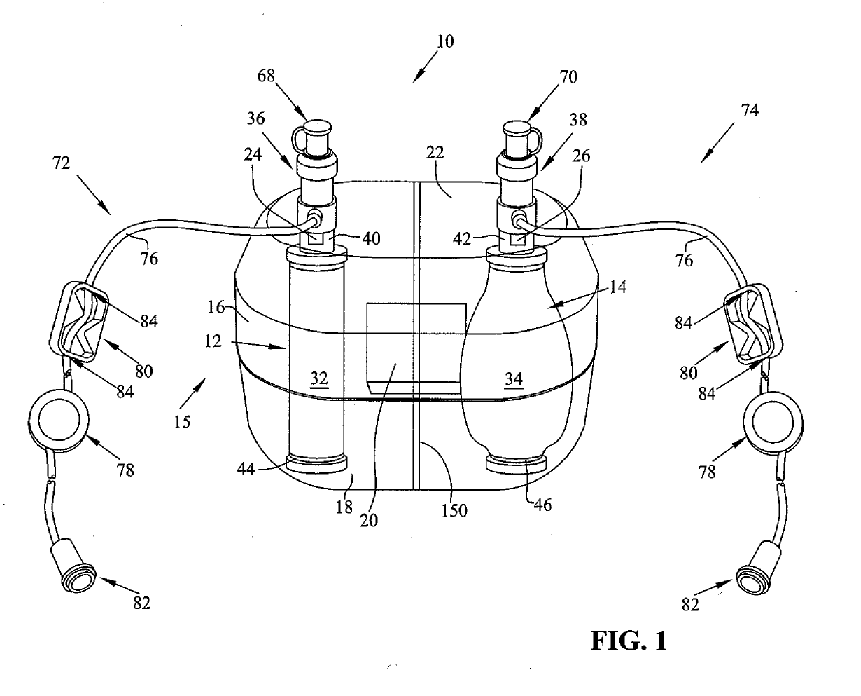

FIG. 1 shows an assembled disposable infusion pump having two discreet reservoirs therein.

FIG. 1 shows an assembled disposable infusion pump 10 having two discrete agent delivery systems therein including reservoirs 12, 14. Reservoirs 12 and 14 are disposed in an interior region of a housing 15 and separated by an internal wall 150. Housing 15 includes upper housing 16 and lower housing 18. Upper housing 16 includes a clasp extension 20 such that when upper housing 16 is coupled to the lower housing 18, clasp extension 20 extends adjacent to lower housing 18 to permit the housing 15 to be selectively coupled to a user's belt, pocket, or the like. Upper housing 16 further includes a top surface 22 having first and second reservoir access voids 24, 26 defined therein. Lower housing 18 includes first and second upwardly extending reservoir attachment posts 28, 30 thereon. In the present embodiment, both upper and lower housings 16, 18 are constructed from plastic that is transparent so as to allow viewing of the first and second reservoirs therethrough.

Each of first and second reservoirs include respective elastomeric balloon reservoirs 32, 34, substance ingress/egress attachments 36, 38, upper reservoir couplers 40, 42, and lower reservoir couplers 44, 46. Balloon reservoirs 32 and 34 are cylindrical elastic members having an axial void defined therein. Attachment posts of respective substance ingress/egress attachments 36 and 38 are received within upper end of respective reservoirs 32, 34 and affixed therein via respective upper reservoir couplers 40 and 42. Similarly, first and second reservoir attachment posts 28, 30 of lower housing 18 are received within the voids of respective balloon reservoirs 32, 34 and coupled thereto via respective lower reservoir couplers 44, 46. Upper and lower reservoir couplers 40, 42, 44 and 46 are ring-type couplers that couple the reservoirs 32, 34 to the respective attachment posts 28 and 30 and ingress/egress attachments 36, 38 by compressing the balloon reservoirs thereto and creating a friction attachment. Ingress/egress attachments 36, 38 each include an internal passageway (not shown) and include fluid ports 160, 162 that allow substances to flow from a position external to attachments 36, 38 into the internal passageway.

Substance ingress/egress attachments 36 and 38 are glued or otherwise affixed within first and second reservoir access voids 24, 26 of upper housing 16. Substance ingress/egress attachments 36 and 38 each respectively include an ingress port extending upward perpendicular to top surface 22 and egress ports extending parallel to top surface 22.

Ingress ports 48, 50 each comprise a threaded cylinder that is compatible with luer lock connectors. Valve housings 56, 58 threadably engage the threaded cylinders of the ingress ports 48, 50 for each of the ingress/egress attachments 36, 38 respectively. Valve housings 56, 58 are configured to have a substance pathway therein that also contains a valve 60, 62 that selectively permits substances to pass. Valves 60, 62 are preferably one-way valves that allow substances to cross the valve 60, 62 in the direction of arrow 500 while preventing substances from crossing in an opposite direction. Upper end of valve housing 56, 58 also includes a luer lock connection suitable for mating with other luer lock connectors. Caps 68, 70 are internally luer lock threaded so as to cap off valve housings 56, 58 when the housings 56, 58 are not otherwise connected to a luer lock connector.

Egress ports 52, 54 are glued or otherwise coupled to capillary tubing sets 72, 74. The sizing of the egress ports 52, 54 and tubing sets 72, 74 determine the maximum flow rate of substances when substances are permitted to flow from reservoirs 12, 14.

Tubing sets 72, 74 each include capillary PVC tubing 76, filters 78, ratchet clamps 80, and luer lock connectors 82. PVC tubing is flexible and has an outer diameter sized to be received within egress ports 52, 54, ports of filters 78, and ports of luer lock connectors 82. Filters 78 are air eliminating filters. Any air that finds its way into tubing 76 between egress ports 53, 54 and filters 78 is not allowed to pass on to tubing 76 downstream of filters 78 but, rather, is expelled through an exhaust port (not shown) in filters 78. Accordingly, once the downstream tubing 76 is primed, air is prevented from entering tubing 76 from upstream. In addition to preventing air from being introduced to a patient connected line, air filter 78 also provides the functionality of preventing backflow of fluids from the patient into tubing 76 once pump 10 has expended its load. Luer lock connectors 82 are connected to mating luer lock connectors (not pictured) on administration devices such as those on needles placed in a patient's skin at desired administration sites.

Clamps 80 provide for starting and stopping flow of agents from their respective reservoirs. Clamps 80 include tubing holes 84 at each longitudinal end for loosely receiving tubing 76 therein. Clamps 80 further include a set of triangular clamp surfaces 86. In an unclamped position, clamp surfaces 86 are spaced apart a distance greater than the outer diameter of tubing 76 such that clamps 80 are loosely and slidably retained upon tubing 76 between egress ports 52, 54 and filters 78. In a clamped position, clamp surfaces 86 are spaced apart a distance less than the outer diameter of tubing 76 such that clamp surfaces 86 engage tubing 76 to distort tubing 76 and prevent flow therein. Clamp surfaces 86 are generally rounded surfaces such that tubing 76 is not cut or otherwise marred when engaged by clamp surfaces 86. Furthermore, tubing 76 has an elasticity such that when clamp 80 is taken from the clamped position to the unclamped position, tubing 76 returns to its normal position with an open inner channel to allow flow therethrough.

In use, caps 68, 70 are removed by a healthcare provider to expose the ingress ports 64, 66 therebelow. The healthcare provider obtains a syringe filled with the desired agent and injects the agent into reservoir 12 across valve 60, 62 and expelling the agent into reservoir 12. As the agent is injected into reservoir 12, balloon reservoir 32 elastically expands to receive the agent therein. By causing balloon reservoir 32 to expand, reservoir is thereby pressurized due to the desire of balloon reservoir 32 to return to a non-expanded rest state. (shown by balloon reservoir 32 in FIG. 1). The healthcare provider may repeat these actions to provide more agent into reservoir 12 if desired. The healthcare provider, by choosing the amount to place in reservoir 12 may select the amount of agent to be delivered. Additionally, by choosing the particular egress port 52 and tubing set 72, the healthcare provider can select the rate at which agent is dispensed from reservoir 12. Accordingly, the healthcare provider may select the amount, rate, and duration of agent delivery within the first agent delivery system.

Reservoir 14 operates similarly to reservoir 12. A healthcare provide may provide the same or a different agent in reservoir 14 than is in reservoir 12. Additionally, the healthcare provider may provide a differently configured egress port 54 and tubing set 34 to provide a different flow rate for the second agent delivery system. Accordingly, pump 10 provides the ability to have two agent delivery systems with distinct reservoirs, distinct agents, distinct dosages, distinct delivery rates, and distinct administration durations that may be applied to distinct administration sites.

It should be appreciated that the pre-fillable and disposable nature of pump 10 allows a patient to receive a prescribed amount of a plurality of agents with pre-defined delivery characteristics. Accordingly, one such pump 10 may be applied to the patient by a healthcare provider and another pump 10 given to the patient for later use. Thus, the patient is permitted to administer agents according to healthcare provider wishes outside of the direct supervision of the healthcare provider. Furthermore, the portable nature of pump 10 also makes operation outside of the direct supervision of the healthcare provider conducive. Permitting patient care outside of the direct supervision of the healthcare provider allows the patient to incur less fees from provider facilities and time and to experience care in a chosen venue rather than the venue of the healthcare provider.

Additionally, the materials from which pump 10 is made are lightweight so as to be conducive to being portable and to be easily attached to a patient's clothing.

Although the invention has been described in detail with reference to certain preferred embodiments, variations and modifications exist within the spirit and scope of the invention as described and defined in the following claims.

1. An infusion pump comprising:
a housing;
a first reservoir disposed within the housing; and
a second reservoir disposed within the housing; the first reservoir being part of a first agent delivery system, the second reservoir being part of a second agent delivery system, the first agent delivery system being discreet and fluidly isolated from the second agent delivery system.

2. The device of claim 1, wherein the first and second reservoir are configured to hold first and second agents therein.

3. The device of claim 2, wherein the first and second agents are different agents.

4. The device of claim 1, wherein the first agent delivery system delivers a first agent at a first rate and the second agent delivery system delivers a second agent at a second rate.

5. The device of claim 1, wherein a first agent is selectively administered via the first agent delivery system independently of whether a second agent is selectively administered via the second agent delivery system.

6. The device of claim 1, wherein the first and second reservoirs are of a type selected from the group including elastomeric balloon-type reservoirs, spring reservoirs, vacuum reservoirs, and any other mechanical reservoir.

7. The device of claim 1, wherein the housing includes a clasp, the clasp being sized and shaped to selectively couple the pump to clothing of a patient.

8. An infusion pump comprising:
a housing;
means coupled to the housing for selectively holding and dispensing a first agent;
means coupled to the housing for selectively holding and dispensing a second agent; and
means for selectively coupling the housing to an article of clothing.

9. The device of claim 8, wherein the first agent holding and dispensing means is fluidly isolated from the second agent holding and dispensing means.

10. The device of claim 8, wherein the first agent holding and dispensing means delivers a first agent at a first rate and the second agent holding and dispensing means delivers a second agent at a second rate.

11. The device of claim 8, wherein the first and second agents are different agents.

12. The device of claim 8, wherein the housing is at least partially transparent to allow a user to view reservoirs of the first and second agent holding and dispensing means.

13. The device of claim 8, wherein the means for selectively coupling the housing to an article of clothing provides for the infusion pump to be worn by a patient such that the patient supports the weight of the pump.

14. A method of providing a plurality of agents to a patient including the steps of:
providing a housing;
providing a first reservoir within the housing;
providing a second reservoir within the housing;
determining a desired egress flow rate for the first reservoir;
determining a desired egress flow rate for the second reservoir;
providing a first tubing set coupled to the first reservoir configured to provide the egress flow characteristics determined in the first reservoir egress determining step; and
providing a second tubing set coupled to the second reservoir configured to provide the egress flow characteristics determined in the second reservoir egress determining step.

15. The method of claim 14, wherein the first reservoir is fluidly isolated from the second reservoir.

16. The method of claim 14, wherein the providing steps cause the first reservoir to deliver a first agent at a first rate and cause the second reservoir to a second agent at a second rate.

17. The method of claim 16, wherein the first rate is different from the second rate.

18. The method of claim 16, wherein the first agent is different from the second agent.

19. The method of claim 14, further including the steps of instructing a user to connect the first and second tubing sets to respective first and second administration devices.
